(19) United States
(12) Patent Application Publication
(10) Pub. No.: US 2020/0345405 A1
(43) Pub. Date: Nov. 5, 2020

(54) HEATED FORCEPS FOR MEIBOMIAN GLAND EXPRESSION

(71) Applicants: Harvey Fishman, Menlo Park, CA (US); Arnold Flores, Menlo Park, CA (US)

(72) Inventors: Harvey Fishman, Menlo Park, CA (US); Arnold Flores, Menlo Park, CA (US)

(21) Appl. No.: 16/398,731

(22) Filed: Apr. 30, 2019

(51) Int. Cl. A61B 18/08; A61B 18/18; A61F 9/007

(52) U.S. Cl. CPC A61B 18/085; A61B 2018/00047; A61F 9/007; A61B 18/1815

(57) ABSTRACT

The heated forceps for Meibomian gland expression disclosed herein may comprise, at least, a forceps and a heating element. The treatment of Meibomian gland dysfunction is benefited by the application of heat to the Meibomian glands during therapeutic manual expression of meibum. The heated forceps for Meibomian gland expression are intended to supply such heat during the manual expression process so as to improve the efficacy of such treatments. The forceps may comprise any appropriate forceps known in the art, and the heating element may be removably attached to the forceps for ease of use and maintenance. In this way the forceps may also be made disposable, while the heating element is reusable.

### TECHNICAL FIELD OF THE INVENTION

The present invention relates in general to medical devices, and, more specifically, to a heated forceps for Meibomian gland expression.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

The Meibomian gland, or the tarsal gland, is a holocrine type of exocrine gland locates at the rim of the eyelid inside the tarsal plate. The gland supplies the eye with meibum, an oily substance that prevents the evaporation of the tear film over the eye and presents the spillage of tears onto the cheek. The meibum traps the eye's tear film between the eye edge and eyeball, and makes the closed eyelids air tight. Dysfunction of the Meibomian gland may present as dry eyes, also called keratoconjunctivitis, and may lead to the development of blepharitis, a condition involving inflammation and crusting of the eyelid.

One common pathology of the Meibomian gland involves its blockage, and is diagnosed as Meibomian gland dysfunction (MGD) or meibomianitis. This blockage results in the failure of the meibum to be secreted into the eye, resulting in the too-quick evaporating of the eye's tear film. Risk factors associated with Meibomian gland dysfunction include advanced age and ethnic background, though any person may be afflicted with the pathology.

Meibomian gland dysfunction presents with a number of common symptoms, including red eyes, grittiness, itchiness, and blurred vision. An ophthalmologist may make the diagnosis of Meibomian gland dysfunction by performing a thorough eye examination, and attempting to express meibum from the Meibomian gland by applying physical pressure to the gland itself. The expression of meibum as a result of pressure may be a direct indicator of the gland's blockage by any number of causes.

Because Meibomian gland dysfunction also affects the stability of the eye's tear film, another diagnostic test that may be attempted is the tear breakup time (TBUT) test. This test involves the application of a small amount of dye to the tear film in front of the eye, and then examination of the dye under cobalt blue light to determine how quickly the tear film breaks down. The faster breakdown time of the tear film is, then, associated with a dysfunction of meibum secretion.

The treatment of Meibomian gland dysfunction primarily involves the application of warm compresses to the eyelids in combination with massage of the eyelids. The goal of such a treatment is to express any meibum clogged within the glands, though these procedures have been shown to rarely treat the cause of the dysfunction. Probing of the Meibomian glands may be done to unclog the glands and attempt to restore normal functioning. The use of antimicrobial eye drops and omega-3 supplements has also been shown to improve the functioning of the Meibomian glands. A more recent approach involving thermal pulsation, which comprises heating the eyelids and applying pulsed pressure to purge the glands, has also been shown to reduce the signs and symptoms of Meibomian gland dysfunction.

A common device used in the applying of manual pressure to the Meibomian glands is the Meibomian gland forceps, which is a forceps having relatively large, rounded surfaces that may fully engage the eyelids and the Meibomian glands. Such forceps may be used in the treatment of Meibomian gland dysfunction to express meibum from the glands themselves. The use of such forceps has been shown to significantly improve patient symptoms over time, and has become a mainstay of first-line treatment for Meibomian gland dysfunction. The use of such forceps is often associated with prior warming or heating of the eyelids to soften the oily buildup within the glands and improve expression of the retained meibum.

Thus, there is a need in the art for a heated forceps for Meibomian gland expression. It is to these ends that the present invention has been developed.

### BRIEF SUMMARY OF THE INVENTION

To minimize the limitations in the prior art, and to minimize other limitations that will be apparent upon reading and understanding the present specification, the present invention describes a heated forceps for Meibomian gland expression.

It is an objective of the present invention to provide a heated forceps for Meibomian gland expression that may comprise a manual forceps.

It is another objective of the present invention to provide a heated forceps for Meibomian gland expression that may comprise a rounded forceps tip.

It is another objective of the present invention to provide a heated forceps for Meibomian gland expression that may comprise a resilient material of construction.

It is another objective of the present invention to provide a heated forceps for Meibomian gland expression that may comprise a flexible material of construction.

It is another objective of the present invention to provide a heated forceps for Meibomian gland expression that may comprise an antimicrobial layer.

It is another objective of the present invention to provide a heated forceps for Meibomian gland expression that may comprise an antimicrobial material of construction.

It is another objective of the present invention to provide a heated forceps for Meibomian gland expression that may comprise a protective coating layer.

It is another objective of the present invention to provide a heated forceps for Meibomian gland expression that may comprise a manual traction layer.

It is another objective of the present invention to provide a heated forceps for Meibomian gland expression that may comprise a heating element.

It is another objective of the present invention to provide a heated forceps for Meibomian gland expression that may comprise a fixedly attached heating element.

It is another objective of the present invention to provide a heated forceps for Meibomian gland expression that may comprise a removably attached heating element.

It is another objective of the present invention to provide a heated forceps for Meibomian gland expression that may comprise a temperature control component.

These and other advantages and features of the present invention are described herein with specificity so as to make the present invention understandable to one of ordinary skill in the art, both with respect to how to practice the present invention and how to make the present invention.

### BRIEF DESCRIPTION OF THE SEVERAL VIEWS OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention.

FIG. 1 is a top isometric perspective view of a heated forceps for Meibomian gland expression having a plug-in heating element, as contemplated by the present disclosure;

FIG. 2 is a top isometric perspective view of a heated forceps for Meibomian gland expression having a plug-in heating element, as contemplated by the present disclosure;

FIG. 3 is a bottom isometric perspective view of a heated forceps for Meibomian gland expression having a plug-in heating element, as contemplated by the present disclosure;

FIG. 4 is a top perspective view of a heated forceps for Meibomian gland expression having a plug-in heating element, as contemplated by the present disclosure;

FIG. 5 is a top isometric perspective view of an exemplary Meibomian gland forceps, as contemplated by the present disclosure;

FIG. 6 is a top isometric perspective view of an exemplary Meibomian gland forceps, as contemplated by the present disclosure;

FIG. 7 is a top isometric perspective view of a plug-in heating element of a heated forceps for Meibomian gland expression, as contemplated by the present disclosure;

FIG. 8 is a top isometric perspective view of a heated forceps for Meibomian gland expression having a portable heating element and pressure sensor, as contemplated by the present disclosure; and

FIG. 9 is a top isometric perspective view of a heated forceps for Meibomian gland expression having a portable heating element and pressure sensor, as contemplated by the present disclosure.

### DETAILED DESCRIPTION OF THE INVENTION

Certain terminology is used in the following description for reference only and is not limiting. The words “front,” “rear,” “anterior,” “posterior,” “lateral,” “medial,” “upper,” “lower,” “outer,” “inner,” and “interior” refer to directions toward and away from, respectively, the geometric center of the invention, and designated parts thereof, in accordance with the present disclosure. Unless specifically set forth herein, the terms “a,” “an,” and “the” are not limited to one element, but instead should be read as meaning “at least one.” The terminology includes the words noted above, derivatives thereof, and words of similar import.

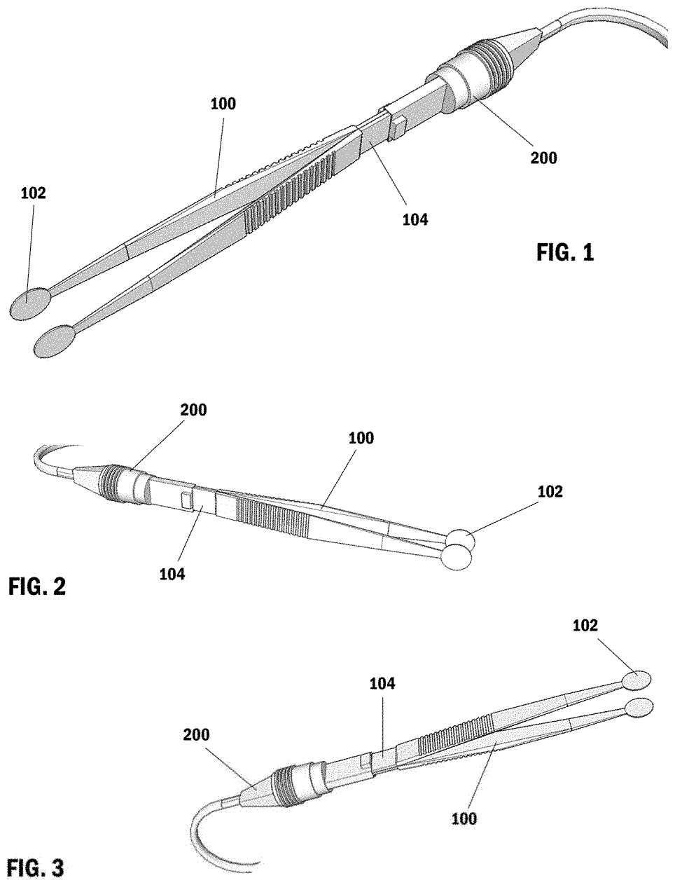

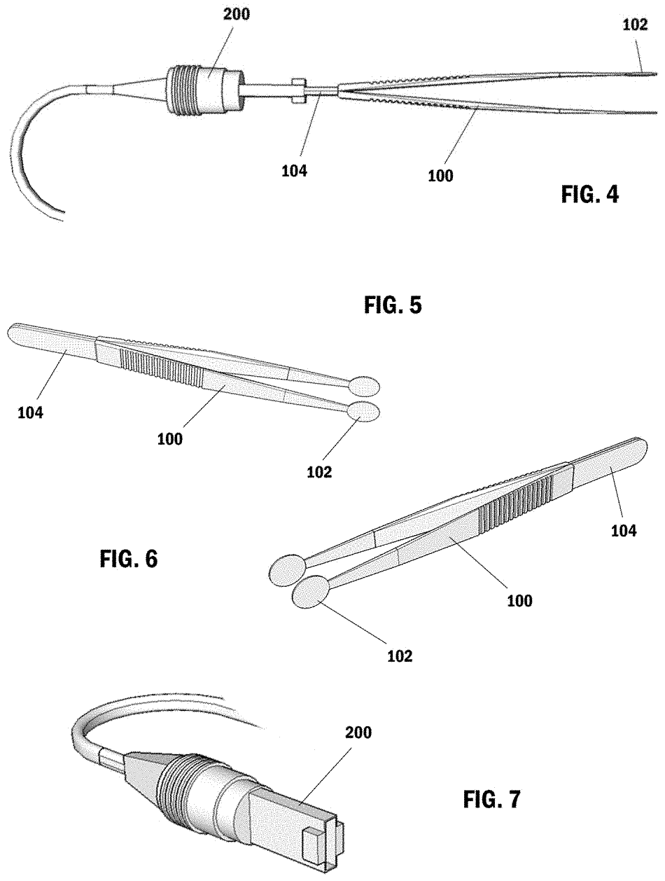

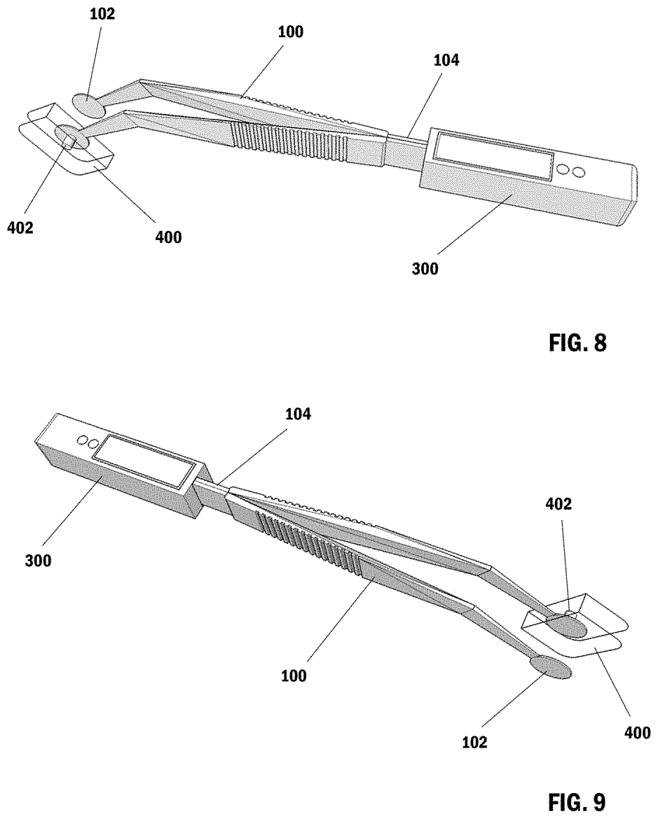

The illustrations of FIGS. 1-9 illustrate a heated forceps for Meibomian gland expression, as contemplated by the present disclosure. The device may comprise, at least, a forceps and a heating element, wherein the forceps may further comprise a plurality of forceps arms 100, a plurality of forceps tips 102, and a forceps base 104. The heating element 200 may comprise a removably or fixedly attached heating element that applies heat to the forceps, and may further comprise a plug-in heating element or a portable heating element.

The forceps may be any style or design of forceps appropriate to the manual manipulation of the Meibomian glands. By way of example, the forceps may comprise a plurality of forceps arms 100 numbering two, connected to one another at a forceps base 104, and being angled away from each other such that they may be pressed towards each other until the plurality of forceps tips 102 are touched together. The plurality of forceps arms 100 may then return to their original, separated orientation wherein the plurality of forceps tips 102 are not touching each other.

The plurality of forceps arms 100 may be any style or design of forceps arms, and may vary in design and construction, as desired. By way of example, the cross-section of the plurality of forceps arms 100 may be rectangular, triangular, circular, or some combination thereof. The length of the plurality of forceps arms 100 may vary, as desired for the particular embodiment, though is generally between three and six inches in length, so as to be easily manipulated while maintaining a small form factor. The plurality of forceps arms 100 may vary in width, as appropriate, for structural rigidity and may be widest at their central portion while tapering to a thinner width nearer the plurality of forceps tips 102. The central portion of the plurality of forceps arms 100 may further comprise indentations on an outer surface for the purpose of traction to the grip of a user of the device. The plurality of forceps arms 100 may further comprise a plurality of holes through their width for the purposes of reducing the weight of the forceps without compromising the structural rigidity of the forceps.

The plurality of forceps arms 100 may further comprise an external covering layer over a part of their length or over their entire length. The external covering layer may be any layer appropriate to provide coating and insulation to the forceps so that a user of the device is protected from the heating of the forceps. The external covering layer may be removably attached to the plurality of forceps arms 100 by, for example, being slid over the plurality of forceps arms 100, or may be permanently affixed to the plurality of forceps arms 100 by, for example, being painted on to the plurality of forceps arms 100. The external covering layer may comprise any appropriate material suitable for providing temperature insulation while also remaining flexible and providing traction to a user of the device.

The plurality of forceps tips 102, which may be attached to a distal end of the plurality of forceps arms 100, may be any size and shape appropriate to the application of manual traction to the Meibomian glands. By way of example, the plurality of forceps tips 102 may be square, rectangular, triangular, circular, or any combination thereof, and may have rounded corners to reduce the chance of cutting or snagging on the soft skin around the Meibomian glands. The plurality of forceps tips 102 may be substantially flat, and may be positioned such that when the plurality of forceps arms 100 are pressed together the flat sides of the plurality of forceps tips 102 abut. In this way the plurality of forceps tips 102 may be used to manually manipulate the Meibomian glands of a user.

The forceps base 104, which may be attached to a proximal end of the plurality of forceps arms 100, may be any size and shape appropriate to the connecting of the plurality of forceps arms 100 to each other. The forceps base 104 may comprise a single piece to which both of the plurality of forceps arms 100 are attached or may comprise two pieces of the plurality of forceps arms 100 attached to each other. The forceps base 104 may further comprise an attachment component to which the heating element 200 may attach and through which heat may be transferred to the forceps.

The heating element 200 may be removably attached to the forceps base 104 or may be fixedly attached to the forceps base 104. In an embodiment wherein the heating element 200 is removably attached, the forceps may be separated from the heating element 200 for more efficient cleaning and sterilization. The heating element 200 may comprise any appropriate heat-generating device with a forceps connection on one end. The forceps base 104 may be installed into the forceps connection and heat from the heating element 200 may then be applied to the forceps itself. In this way, the forceps, which may be made from a heat-conducting material, are heated by the heating element 200, and this heat may be applied to the Meibomian glands.

The heating element 200 may further comprise a power source or connection to a power source for supplying power to the system. The heating element 200 may further comprise a means for controlling power delivery to the heat source of the device and, thus, controlling the temperature of the forceps connected to the device. In one embodiment the heating element 200 may comprise a plug-in heating element having a forceps connection at one end, a central control box, and an alternating current (AC) plug at the other end. In a second embodiment the heating element 200 may comprise a portable heating element having a forceps connection at one end, a central control box, and a battery power source. The battery power source may be rechargeable or may carry a fixed charged for limited use.

The heating element 200 may further comprise a temperature control device for monitoring and controlling the amount of heat applied to the heated forceps for Meibomian gland expression. The temperature control device may be an analog or digital device or display, and may relate relevant information to a user of the forceps. Such information may include, for example, temperature, temperature control options, battery level, battery power, and any other relevant information. The temperature control device may further comprise an audible alarm or vibrational alarm that may alert a user of the device to overuse, the settings for which may be adjustable.

In one embodiment the heating element 200 may comprise a means for induction heating, or magnetic heating, through electromagnetic radiation from heat generated by eddy currents. Such an embodiment may further comprise an electromagnet and an electronic oscillator driving alternating current through the electromagnet. The magnetic field created by the driving of the current may cause the forceps material to heat up as desired.

In another embodiment the heating element 200 may comprise a means for conductive heating using temporarily or permanently applied magnetic paint that allows for heating.

In another embodiment the heating element 200 may comprise a means for radiant heating using light-emitting diodes (LEDs).

In another embodiment the heating element 200 may comprise a light-absorbing paint that may be illuminated and heated with a laser source.

In another embodiment the heating element 200 may comprise a means for microwave heating between the plurality of forceps arms 100.

In another embodiment the heating element 200 may comprise a plurality of silver-ink and polyethylene terephthalate (PET) micro-heater sheets on the plurality of forceps arms 100.

In another embodiment the heating element 200 may comprise a means for applying the Peltier effect.

In another embodiment the heating element 200 may comprise a means for chemically-induced exothermic heating within the forceps assembly.

In another embodiment the heating element 200 may comprise the application of heated water over the plurality of forceps arms 100.

In another embodiment the heating element 200 may comprise a plurality of forceps arm cover sleeves comprising an exothermic material.

In another embodiment the heating element 200 may comprise a means for heating one of the plurality of forceps arms 100 while not heating the other.

In another embodiment the heating element 200 may be made from an exothermic material of construction.

The heated forceps for Meibomian gland expression may further comprise a pressure control device 300, which itself may comprise a pressure sensor 302. The pressure control device 300 may be installed over a forceps tip 102, and may relate information relevant to the amount of pressure being applied by a user of the device. The pressure control device 300 may be an analog or digital device or display, and may relate relevant information to a user of the forceps such as, for example, pressure, application time, battery level, battery power, and any other relevant information. The pressure control device 300 may further comprise an audible alarm or vibrational alarm that may alert a user of the device to overuse, the settings for which may be adjustable.

The heated forceps for Meibomian gland expression may further comprise a vibration control device, which may induce a selectable vibration in the plurality of forceps arms 100. The vibration control device may allow for the vibration to be adjusted, as desired, to increase or decrease the vibration frequency of the heated forceps. The vibration control device may be a vibrating motor or micro-motor, which may be built into the heating element 200 of the device, and which may be activated to create vibrations that assist with the breakup up meibum plugs.

The heated forceps for Meibomian gland expression may further comprise a plurality of fiber optic wiring run through the plurality of forceps arms 100 such that a light source may be focused through the fiber optic wiring and emitted through the end of the plurality of forceps arms 100. In this way the heated forceps may provide a light source to illuminate an area being worked upon.

The heated forceps for Meibomian gland expression may further comprise a plurality of removable or disposable tip covers. Such tip covers may be removably installed onto the forceps tips 102 by any appropriate means such as, for example, slip-fit, wrapping, or any other means. The tip covers may fit tightly over the forceps tips 102 so as not to interfere with the functioning of the heated forceps for Meibomian gland expression, and may serve to prevent the known risk of prion transmission from one patient to the next.

The heated forceps for Meibomian gland expression may be substantially constructed of any suitable material or combination of materials, but typically is constructed of a resilient material or combination of materials such that the device is resistant to damage as a result of compression, twisting, heating, or submersion in water. As an example, and without limiting the scope of the present invention, various exemplary embodiments of the heated forceps for Meibomian gland expression may be substantially constructed of one or more materials of plastic, acrylic, polycarbonate, steel, aluminum, brass, fiberglass, carbon fiber, or combinations thereof. In some embodiments the various components of the device may be coated, lined, or otherwise insulated to prevent contamination of the device. In one embodiment the material of construction may vary from one component to the next within the system.

In one embodiment the heated forceps for Meibomian gland expression may comprise a resilient material of construction that either comprises a material having antimicrobial properties or comprises a layering of antimicrobial material or coating. Antimicrobial properties comprise the characteristic of being antibacterial, biocidal, microbicidal, anti-fungal, anti-viral, or other similar characteristics, and the oligodynamic effect, which is possessed by copper, brass, silver, gold, and several other metals and alloys, is one such characteristic. Copper and its alloys, in particular, have exceptional self-sanitizing effects. Silver also has this effect, and is less toxic to users than copper. Some materials, such as silver in its metallic form, may require the presence of moisture to activate the antimicrobial properties.

While the invention has been described in connection with what is presently considered to be the most practical and preferred embodiments, it is to be understood that the invention is not to be limited to the disclosed embodiments, but, on the contrary, is intended to cover various modifications and equivalent arrangements included within the spirit and scope of the appended claims.

I claim:

1. A heated forceps for Meibomian gland expression, comprising:
a forceps; and
a heating element;
wherein said forceps further comprise a plurality of forceps arms, a plurality of forceps tips, and a forceps base; and
wherein said heating element is attached to said forceps.

2. The invention of claim 1,
wherein said heating element is attached to said forceps base of said forceps.

3. The invention of claim 1,
wherein said heating element is removably attached to said forceps.

4. The invention of claim 1,
wherein said heating element is fixedly attached to said forceps.

5. The invention of claim 1,
wherein said heating element comprises a power supply.

6. The invention of claim 1,
wherein said heating element comprises a temperature control device.

7. The invention of claim 1,
wherein said heating element comprises a plug-in device.

8. The invention of claim 1,
wherein said heating element comprises a portable device.

9. The invention of claim 1,
wherein said heating element comprises a user-defined alarm.

10. The invention of claim 1,
wherein said forceps comprises a flexible material.

11. The invention of claim 1,
wherein said forceps comprises a rigid material.

12. The invention of claim 1, further comprising:
an external covering layer.

13. The invention of claim 12,
wherein said external covering layer comprises a heat-resistant material.

14. The invention of claim 12,
wherein said external covering layer comprises a flexible material.

15. The invention of claim 1, further comprising:
a pressure control device.

16. The invention of claim 15,
wherein said pressure control device comprises a pressure sensor.

16. The invention of claim 15,
wherein said heating element comprises a user-defined alarm.

17. The invention of claim 1, further comprising:
a plurality of fiber optic wires.

18. The invention of claim 17, further comprising:
a light source;
wherein said light source emits light into said plurality of fiber optic wires.

19. The invention of claim 1, further comprising:
a vibrating mechanism.
